(19) United States
(12) Patent Application Publication
(10) Pub. No.: US 2008/0067123 A1
(43) Pub. Date: Mar. 20, 2008

(54) Substrates With Modified Carbon Surfaces

(76) Inventors: James D. Burrington, Mayfield Village, OH (US); Kirk E. Davis, Chesterland, OH (US); Scott T. Jolley, Mentor, OH (US); Ralph E. Kornbrekke, Chagrin Falls, OH (US); Joseph W. Pialet, Euclid, OH (US); Philip W. Pike, Concord Township, OH (US); Roger L. Sowerby, Concord Township, OH (US)

Correspondence Address: THE LUBRIZOL CORPORATION;ATTN: DOCKET CLERK, PATENT DEPT., 29400 LAKELAND BLVD., WICKLIFFE, OH, 44092, US

(21) Appl. No.: 11/943,998

(22) Filed: Nov. 21, 2007

Related U.S. Application Data
(63) Continuation of application No. 10/478,935, filed on May 8, 2004.
(60) Provisional application No. 60/295,316, filed on Jun. 1, 2001.

(51) Int. Cl. C02F 1/26; C07C 233/00; C07C 55/02; C07C 59/00; C07C 69/34; C07C 69/66; C07D 307/34

(52) U.S. Cl. 210/633; 549/253; 560/174; 560/190; 562/577; 562/590; 564/197

(57) ABSTRACT

A process for functionalizing a carbon surface and the product thereof is disclosed. The first reactant used contains one or more electron withdrawing groups that thereafter can be reacted with other compounds. The reaction product has enhanced dispersability, interaction with other media, or other utilitarian uses, e.g. a reactive surface. The reaction product is then incorporated into an elastomeric or thermoplastic composition; it can be dispersed in a liquid media such as an ink, coating, or lubricant; or it can be used as a solid in applications such as a filtering media.

### FIELD OF THE INVENTION

Substrates with carbon surfaces such as carbon black, carbon fibers, graphite, and activated carbon are modified by reacting with a reactant having at least one double bond and one or more electron withdrawing groups, e.g. maleic anhydride. This reaction is anticipated to create significantly more reactive groups (relatively) on the carbon surface that can be functionalized (if desired) by having a nucleophilic group in the functionalizing agent. These functionalized materials can be used in composites, filtration media, coatings, inks, etc.

### BACKGROUND OF THE INVENTION

Carbon black and related substrates having surfaces rich in carbon, e.g. graphite, and carbon fibers, have been considered as relatively non-reactive with most chemical compounds. Silica surfaces which, are rich in hydroxyl groups (also called silanol groups), have been functionalized with a variety of reactants to enhance the interaction of silica with a variety of continuous media such as elastomers and solvents. Some research has been conducted on oxidizing carbon rich surfaces to create carbonyl and carboxy groups on the surface, which would react with nucleophiles. While this work was successful at oxygenating the surface, the physical properties of the substrates were typically also modified during the reactions at elevated temperatures or using strong oxidants such as peroxide or ozone.

### SUMMARY OF THE INVENTION

Carbon Rich Surfaces can be Reacted with Compounds of Formula I:

X₁X₂C═CX₃X₄ or Formula II: X₁C(H)═O; wherein X₁, X₂, X₃ and X₄ are independently selected from H, an alkyl of 1 to 4 carbons, or an electron withdrawing group, wherein at least one of X₁, X₂, X₃ and X₄ is a known electron withdrawing group, said electron withdrawing groups being characterized by having a σ, >0. where σₚ is the log(K′/K₀′) where k₀′ is the equilibrium constant for the ionization of a para substituted benzoic acid with the particular group and k₀′ is the equilibrium constant for the ionization of benzoic acid in water at 25° C., under conditions such as elevated temperatures to effectively bond a significant portion of the compounds of Formula I or II to the carbon surface. This effectively places X₁, X₂, X₃ and/or X₄ on the carbon surface where they can serve as a point of chemical bonding with nucleophilic reactants. Preferred reactants are maleic acid or anhydride, methyl acrylate, itaconic acid, acrylic acid, glyoxylic acid, the hemiacetal of the methyl ester of glyoxylic acid, and methyl glyoxylate.

Depending on the particular application (embodiment) desired the first reaction product can be further reacted with nucleophilic compound(s) that carry or contain particular reactive groups for further reaction or utility.

A significant application can be a filler in a elastomeric or thermoplastic composite where the carbon rich surface is part of a carbon based filler for the elastomer or thermoplastic. Other applications include dispersing the substrate with a carbon surface in an ink, coating, or a lubricant; or using the substrate with a carbon surface as a filtering media.

### DETAILED DESCRIPTION OF THE INVENTION

The carbon surface can be any form of condensed carbon. The substrate supporting the carbon surface can also be carbon or carbon based or it can be another support material. In most embodiments the substrate is substantially the same material as the surface, e.g. carbon black, graphite, carbon fibers, activated carbon. Desirably the carbon surface must be at least one molecular layer thick if the entire substrate is not carbon. In most embodiments the carbon on the surface constitutes at least 75 weight percent, more desirably 80 and preferably 85 weight percent of the surface. If the substrate is to be carbon based desirably at least 50 weight percent, more desirably 70, and preferably at least 80 weight percent of the substrate is carbon.

A general listing of condensed carbon includes particulate carbon such as carbon black and soot, graphite, diamond, carbon fibers, activated carbon, charcoal, activated charcoal, carbonized surfaces e.g. partially carbonized coconut shells, carbon nanotubes, carbon nanoparticles, graphitic nanoparticles, and carbon-containing fullerenes, such as C60. The carbon can be amorphous, crystalline, or a mixture of amorphous and crystalline. The crystalline portion can be three-dimensional crystals, such as diamond, or two-dimensional crystals, such as graphite. The carbon if it constitutes a major portion of substrate can be particulate, granules, chunky, fibers, or rods (e.g. anodes) etc.

The carbon surface is desirably treated with a reactant of Formula I: X₁X₂C═CX₃X₄ or Formula II: X₁C(H)═O wherein X₁, X₂, X₃ and X₄ are independently selected from H, an alkyl of 1 to 4 carbons, or an electron-withdrawing group, wherein at least one of X₁, X₂, X₃ and X₄ is a known electron-withdrawing group, said electron withdrawing groups being characterized by having a σₚ>0. where σₚ is the log(K′/k₀′) where k′ is the equilibrium constant for the ionization of a para substituted benzoic acid with the particular group and k₀′ is the equilibrium constant for the ionization of benzoic acid in water at 25° C. Desirably the molecular weight of each of the electron-withdrawing groups X₁, X₂, X₃ and X₄ is less than 100 grams/mole and the molecular weight of the entire molecule of Formula I or II is less than 400 and more desirably less than 200 grams/mole.

Preferred molecules for Formula I are maleic acid, maleic anhydride, alkyl or alkenyl substituted maleic acid or anhydride, and the diels-alder adduct of dienes or polyenes with maleic anhydride or maleic acid, such as nadic anhydride or nadic methyl anhydride. Alternatively it can be acrylic acid, methacrylic, other C₂-C₄ allyl substituted acrylic acid, itaconic acid, or C₁-C₄ substituted itaconic acid, or C₁-C₆ alkyl esters or partial esters of the specified acids.

Preferred molecules for Formula II are glyoxylic acid or esters thereof, derived from reacting glyoxylic acid and C₁-C₄ alcohols and the hemiacetals of C₁-C₄ alkyl esters of glyoxylic acid.

X₁, X₂, X₃ and X₄ are desirably selected from carboxylic acid, C₁-C₁₀ esters and salts of carboxylic acids. When Formula I is an anhydride of dicarboxylic acids, two of X₁, X₂, X₃ and X₄ combine to form the anhydride. X₁, X₂, X₃ and X₄ call also be or contain ester, amide, nitrile, nitro, keto, and aldehyde groups.

To make the reaction product one applies the reactant of Formula I or II to the surface of the carbon and applies heat. The reaction can be carried out neat using any gaseous environment such as air or inert gas (e.g. argon or nitrogen), or using a liquid solvent (either polar e.g. water or nonpolar), optionally with catalysts present to promote a faster or more effective chemical reaction between the reactant of Formula I or II with the carbon surface. Typical catalysts are Lewis (e.g., BF₃) or Bronsted (e.g., H₂SO₄) acids.

A preferred method is to apply the compound of Formula I or II rather uniformly to the carbon surface by a spray addition, metering, or bulk addition (optionally mixing to further disperse) and then heat the carbon surface and reactant for a few seconds or minutes to several hours at a temperature from about 60° C. to about 500° C. and preferably from about 100 to 350° C., and most preferably from about 150 to about 300° C. Desirable reaction times are from a few seconds or minutes to one or more days (24 hours or more), depending on the reaction temperature.

As a more efficient process, the carbon black and Formula I or II are fed into a heated zone of a mixing vessel as an aerosol; Formula I or II vaporizes and reacts with the carbon black at temperatures between 200 and 500° C., more preferably 350 to 450° C. for seconds to minutes of resonance time in the heated zone. The carbon black could be any commercially produced material, or it could be a stream from the carbon black production process—while it is still very hot and before being quenched. The heated zone could be in a continuous feed reactor, and the ratio of carbon black to Formula I or II might be similar to those used in the batch process with excess Formula I or II—so weight ratios of Formula I or II to carbon black of 1:1 to 1:20, more preferably between 1:5 and 1:10, depending upon the adjustments in residence time and temperature and desired characteristics of the product.

The reaction product of a reactant of Formula I or II with a carbon surface can be characterized with (photoacoustic) infrared analysis (PA-FTIR), solid state proton NMR, X-Ray Photoelectron Spectroscopy (XPS), solvent extraction, and/or thermogravimetric analysis. It is generally observed that new infrared peaks and NMR peaks appear after the reaction, a significant portion (usually not all) of the reactant of Formula I or II is no longer extractable with appropriate solvent extraction techniques and the reaction product, when tested by thermogravimetric analysis, loses weight at different (higher) temperatures than a simple blend of the reactant of Formula I or II and the same carbon surface. These analyses indicate that some form of chemical reaction or physical interaction has occurred between the carbon surface and the reactant.

The carbon surface treated with a reactant of Formula I or II can be further reacted with a nucleophile of the formula R′-Nu, where R′Nu contains one or more nucleophilic group(s) known to react with the electron withdrawing groups of X₁, X₂, X₃ and/or X₄. Said nucleophilic groups include NH₂, NHR, NR₂, OH, SH, SR, PR₃ (or —PR₂), P(OR)₃ (or —OP(OR)₂), NRNHR, NRNR₂, NROR and OOR or any anionic form thereof where R and R′ are independently hydrogen, a hydrocarbyl group (optionally being a polyether or polyamine group), a cation containing group, a di, tri or polysulfidic linkage, or combinations thereof. A preferred R′-Nu is ethylene diamine or a polyamine derived from ethylenediamine such as H—[NH—CH₂—CH₂], —NH₂ where n=2-6. A broader group of R′-Nu would be the alkylene polyamines represented by the formula R—N(R)— (Alkylene-N(R))ₙ—R where n can vary from 1-7 or 1-10, each R is independently a hydrogen atom, a hydrocarbyl group or a hydroxy-substituted hydrocarbyl group having up to 30 or 50 carbon atoms, and “alkylene: refers to 1-6 or 1-18 carbon atoms in a linear or branched form. Commercial products of these formulas include these structures along with variants thereof. For example E-100 from Dow Chemical Company of Freeport, Tex. has about 22% tetraethylenepentamine and 77% pentaethylenehexamine. A Union Carbide product known as HPA-X® includes cyclic condensation products along with higher analogs of diethylenetriamine and triethylenetetramine. The nucleophile can also be a polyether and potassium hydroxide. This is a preferred nucleophile in some embodiments when Formula I is maleic acid or anhydride. The nucleophile can be a coupling aid or agent between the substrate with a carbon surface and another chemical compound e.g. elastomer, plastic, solvent, carrier etc. A coupling aid or agent is generally defined as a material that has two attractions or can chemically or physically bond to two different materials together. Generally it is anticipated that one nucleophilic portion of the nucleophilic compound will be attracted or chemically bond to one of X₁, X₂, X₃ and/or X₄ on the carbon surface and another portion of the nucleophilic compound (if it is functioning as a coupling aid) will be attracted to or bond to another chemical material, e.g. an elastomer, plastic, solvent etc.

As used herein, the term “hydrocarbyl substituent” or “hydrocarbyl group” is used in its ordinary sense, which is well known to those skilled in the art. Specifically, it refers to a group having a carbon atom directly attached to the remainder of the molecule and having predominantly hydrocarbon character.

Examples of Hydrocarbyl Groups Include:

(1) hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form an alicyclic radical);

(2) substituted-hydrocarbon substituents, that is, substituents containing non-hydrocarbon groups which, in the context of this invention, do not alter the predominantly hydrocarbon substituent (e.g., halo (especially chloro and fluoro), hydroxy, alkoxy, mercapto, alkylmercapto, nitro, nitroso, and sulfoxy);

(3) hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in the hydrocarbyl group.

The substrates with a carbon surface can be used as a media or as a filler (optionally a reinforcing filler) in a variety of applications. The reactant of Formula I or II could be characterized as containing one or more electron-withdrawing groups. The electron withdrawing groups X₁, X₂, X₃ and X₄ create additional reactive sites on the carbon surface without requiring harsh conditions that are typically required to oxidize carbon surfaces in the absence of a reactants The relatively mild reaction conditions under which the reactant of Formula I or II is added to the carbon surface allows chemical modification of the carbon surface while minimizing changes in the properties below the carbon surface (i.e. in the substrate). The properties below the carbon surface may include things like morphology, particle size, porosity, density, crystallinity, and the presence or absence of heteroatoms other than carbon and hydrogen. It would be desirable to leave all of these substrate properties unchanged while adding the reactants of Formula I or II to the carbon surface of a substrate.

Of particular interest is an embodiment where the substrate with a carbon surface is used as a filtration media to 1) remove a component, 2) add a component, or 3) exchange a component from a media. In this particular embodiment a liquid media would include a gaseous material or a flowable solid (liquid-measure type). The substrate with a carbon surface could be a particulate carbon such as carbon black or a larger size substrate such as activated carbon or charcoal. The substrate could be suspended in a media or used as a packed bed, column or filter media. The reaction product of the carbon surface and the reactant of Formula I or II could be the chemically active part of the filtration media or the reaction product can be reacted with a nucleophilic compound as described above to add another functional group to the filtration media.

Another embodiment is to use the modified carbon surface and its corresponding substrate, either modified only with the reactant of Formula I or II, or further reacted thereafter with a nucleophilic compound to 1) aid in the dispersability of the substrate with a carbon surface in another media or 2) change the interaction of the carbon surface with the media after being dispersed (either physical or chemical interaction with the media, e.g. an elastomer, plastic, solvent etc.). In these applications the carbon surface modification would somehow make the substrate with the carbon surface more dispersible or more effective at interacting with another media such as the continuous media or other dispersed media. Applications include using the substrate with a carbon surface in an ink, coating, fiber-reinforced plastic, compounded elastomer, compounded plastic, etc to make an improved product. Some of these compositions would desirably be water-based for environmental reason, such as inks and coatings, while others might be use solvents other than water. If the substrate with a carbon surface is a fiber, then the reaction product of the substrate with a carbon surface, the reactant of Formula I or II and optionally further reacted with a nucleophilic compound can be one of the fiber component(s) of a fiber-reinforced composite. It is possible for the reactant of Formula I or II and/or its subsequent reaction product with a nucleophilic compound to function both as a dispersing and a coupling aid in some situations.

In elastomer compositions formulated in the rubber industry it is common to specify the ingredients based on parts by weight per 100 parts by weight of rubber (phr). A very common form of rubber is derived from polymerizing conjugated diene monomers having from about 4 to 8 carbon atoms and optionally up to one heteroatom per monomer, such as isoprene, butadiene, or chloroprene. Sometimes a conjugated diene monomer such as butadiene is copolymerized with at least one other monomer such as styrene to form a copolymer, terpolymer etc. depending on the number of comonomers. Natural rubber is a rubber derived from polymerizing isoprene. Generally to get elastomeric properties from such polymers with significant amounts of repeating units from conjugated dienes, it is desirable to have at least 30 weight percent of the repeating units derived from a conjugated diene, more desirably at least 40 weight percent. Substrates having carbon surfaces, e.g. carbon black, are generally used in elastomers compositions at concentration above 1 phr, and more desirably above 20 phr and preferably above 30 phr. Coupling agents or aids (couplers) that potentially can enhance the interaction between the rubber and the carbon surface are generally used above the 0.5 phr concentration and more desirably above 1 phr. A preferred coupler would desirably have an amine group to react with X₁, X₂, X₃ and/or X₄, and either a thiol or polysulfidic linkage that might couple to unsaturation in a rubber compound. A more preferred coupler would also have a carbonyl group such as an ester or amide linkage. Such a coupling agent could be formed from a first amino compound and a second compound, said second compound having both a group that can couple through a condensation reaction with said first amino compound and another group being a thiol, which can, optionally, be converted to a polysulfidic linkage. Synthesis of such a compound is disclosed in copending World application U.S. Ser. No. 01/09290 filed Mar. 22, 2001, which is hereby incorporated by reference for its teachings on thiol or polysulfidic compound that might be used as coupling aids. A preferred second compound is 3-thioproprionic acid methyl ester, where the resulting coupling agent can possibly be reacted with elemental sulfur to form a second coupling agent with a polysulfidic linkage in place of the thiol group.

A desirable amount of the above described coupling agent(s) is generally from about 0.07 to about 300 parts by weight per 100 parts by weight of carbon, more desirably from about 0.5 to about 10 parts by weight per 100 parts by of weight of carbon. The amount of coupling agent generally will vary with the amount of carbon surface area per gram, which can vary significant, depending on whether one is describing a high surface area carbon black or a low surface area graphite.

### EXAMPLES

### Examples 1-7

Treated carbon black N234, Carbon black and solid maleic anhydilde (or another reactant compound with electron-deficient unsaturation) are reacted, either neat or in a slurry of an appropriate solvent, at a temperature and time such that a substantial amount of free maleic anhydride has become reacted or otherwise strongly bonded to the carbon black. In an embodiment with simple stirred reactors temperatures are generally 100-250° C., more preferably 150-230° C. Preferred times are 1 to 36 hours, more preferably, 12 to 24 hrs. After reaction, the solids are washed with acetone and filtered to remove any free reactant. Thus the amount of reactant left on the solid form of carbon should be based on the % wt. reactant column in TABLE 1 rather than the treat rate. Each of the Examples 1-11 showed 3 thermogravimetric analysis features at about 200° C., 320° C., 550° C., which are not present in the carbon controls or samples which had been impregnated with maleic anhydride in solvent and dried, but not heated. The latter gave a large peak at 66° C., which is not present in any of the samples from Ex 1-6. These products also contained more surface oxygen (based on XPS analysis) than the carbon black starting materials. The solid state proton NMR spectrum of this product has a broad peak between 2-3 ppm, and this is what would be expected for the reaction of maleic anhydride with a double bond on the carbon black—however there are several possible products for this reaction. The PAS-FTIR spectrum shows a peak at 1805 cm₋₁, and this is the peak associated with the maleic anhydride carbonyl; hence the product contains this type of functional group.

The specific reactants and conditions are shown in Table 1 below. N234 is available from Engineered Carbons, Inc in Borger, Tex., and has a reported particle diameter of 21 nm, a nitrogen surface area of about 125 m²/g and is used for high reinforcement in rubber compounds. The graphite used was Graphite 3442, a graphite flake from Asbury Graphite Mills, Inc having 99 wt. % passing through a 325 mesh screen.

TABLE 1
Reactions of Carbon Black (CB) and Graphite (G) with Maleic Anhydride (MA),
Itaconic Anhydride (IA), Succinic Anhydride (SA) or Methacrylic Acid (MAA).

| | | Reactant (wt/wt | | | | % wt |
|---|---|---|---|---|---|---|
| Ex. No. | Carbon | Carbon) | Temp, C. | hr | % O^{a} | reactant^{b} |
| Control A | N234 CB | none | (Control for Ex 1-2, | | 2.5 | 0 |
| | | | 4-7, 9-11 | | | |
| Control B | G 3442 | none | (Control for Ex 3) | | 4.1 | 0 |
| Control C | N234 CB | none | (Control for Ex 8) | | 1.6 | 0 |
| 1 | N234 CB(f) | MA(1/10) | 200 | 24 | 4.2 | 4.6 |
| 2 | N234 CB | MA(1/10) | 200 | 24 | 5.0 | 6.8 |
| 3 | G 3442 | MA(1/10) | 200 | 24 | 5.4 | 3.5 |
| 4 | N234 CB | MA(1/100) | 200 | 24 | — | — |
| 5 | N234CB | MA(1/5) | 200 | 24 | 7.1 | 12.5 |
| 6 | N234CB | IA(1/10) | 200 | 24 | 4.4 | 5.2 |
| 7 | N234CB | MA(1/20) | 200 | 24 | 3.4 | 2.5 |
| 8 | N234CB | MA(1/1, in Cl₃C₆H₃) | 200 | 12 | 4.5 | 7.8 |
| 9 | N234CB | MA(1/10) | 93 | 12 | 2.7 | 0.5 |
| 10 | N234CB | MA(1/10) | 200 | 14 | 4.8 | 6.3 |
| 11 | N234CB | MA(1/4) | 200 | 22 | 8.4 | 16.1 |
| 12 | N234CB | MAA(1/4) | 200 | 22 | — | 3.5^{d} |
| 13 | N234CB | SA(1/4) | 200 | 22 | — | 2.5^{d} |

(^{a}= atomic %, by XPS);
^{b}calculated from % O;
^{c}(f) = fluffy CB; otherwise, pelletized CB);
^{d}% wt by gravimetric analysis.

### Example 14-17

(TABLE 2) These samples were prepared by aqueous reaction of the selected treated carbons from TABLE 1 with nucleophiles. The resulting solid was then filtered, washed (water), and dried.

TABLE 2
Reactions of Maleic Anhydride-treated Carbon Black with
Nucleophiles.

| | Treated-C | Nucleophile | Reaction | Reaction | | |
|---|---|---|---|---|---|---|
| Ex. No. | Ex No. | (mCO:mN) | Temp. | Time hr | % O* | % N* |
| 14 | 11 | H—(NHCH2CH2NH)x—H | 110° C. | 6 | 4.8 | 5.5 |
| | | (˜0.3) | | | | |
| 15 | 11 | NH2NH2 (0.5) | 80 | 6 | 7.0 | 1.4 |
| 16 | 11 | NH2OH (1) | 50 | 1 | 7.4 | 0.8 |
| 17 | N/a | H₂O₂ | 10 | 3 | 7.7 | |

(*atomic % by XPS)

Example 17 was prepared with a carbon black reacted with maleic anhydride under different conditions than examples 1-13. The material of Example 17 was made up to determine if peracid groups could be attached to the carbonyl functionalized carbon black. Example 17 illustrated that this was possible but the reaction temperature was desirably low so that the peracid doesn't decompose.

### Examples 18

### Preparation of Carbon Coupler

A coupler was prepared by reaction of 3-mercaptopropionic acid methyl ester with an excess of ethylene diamine at 30° C. at for 1 hr to give a quantitative yield of the corresponding 1:1 mole ratio mercapto-amino amide (after removing unreacted ethylene diamine) by IR and elemental analysis, 20.3 S % (21.9% theory); 19.1% N (18.89% theory). Two moles of this material was reacted with 3 moles of elemental sulfur at 100 C for 4 hr to give a 94.5% yield of a dark red glass, with IR and elemental analysis (32.7% S, 37.71% theory; 16.6% N, 15.63% theory) consistent with the corresponding amino amide polysulfide (EDA) of the structure:

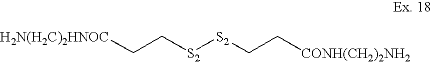

### Example 19

CB/Malan pre-reacted with coupler (EDA) of Ex18. To MAA/CB from Ex 8 (410 g, corresponding to 18 g, 0.184 mole Malan), suspended in toluene, was added material from Ex 18 (31.3 g, 0.184 mole) in 20 ml of water with stirring. The reaction was refluxed until no more water was removed (about 23 ml), and the solid washed with toluene and dried to give a solid, 431 g,

### Examples 20-26

Rubber formulations using various forms of carbon black are shown in TABLE 3. CB/no coupler is generally a control without maleic anhydride-or coupler (see Control for examples 20-26, Control for example 25, and Control for example 26). Multiple controls were used because the scorch time and/or the cure rate of the rubber compounds varied depending on the additional treatments to the carbon black. Therefore additional controls were run to reflect changes in the mixing procedures to compensate for different effective cure rates of the various rubber compounds. The Control for examples 20-26 is believed to have been an oxidized N234 with a slower cure rate. Pre-reacted C/MA/Ex 18 refers to the product of example 19 where maleic anhydride-treated carbon black was reacted with the coupler from Ex 18 in toluene. Since example 19 included 4 phr of coupler and 80 grams of carbon black it was added at 84 phr with no additional coupler. Pre-treat means that an aqueous solution of the coupler (Ex 18) was adsorbed onto the carbon black/maleic anhydride, followed by drying, prior to its addition to the rubber. Examples 21, 22, 23, and 25 show pre-treat with level of coupler going from 3 to 5 phr. In the “dry-mix” mode, the carbon black/maleic anhydride material was substituted for carbon black (@ 80 phr) and added directly, along with other coupler (4 phr of coupler), during rubber mixing (Ex 26). Example 24 uses CB/MAA (from Ex 8) without coupler. Addition of carbon black/maleic anhydride, coupler or pre-reacted coupler w/MA/CB (Ex 19) can be added in one portion, split over time, or over stages of the rubber mixing.

Rubber Composition for Examples 20-26:

| Component | Level (PHR) |
|---|---|
| Duradene 715 S-SBR | 70 |
| Budene 1207 high cis BR | 30 |
| N234 carbon black/MAA (or CB - control) | 80 |
| Couplant (specified in Table 3) | 0-5 |
| Textracts 2202 aromatic oil | 36 |
| ZnO - Stearic acid | 3-2 |
| Sulfur - CBS accelerator | 1.5-1.5 |
| Flexzone 7P antioxidant | 1 |

Duradene 715 is a solution polymerized styrene-butadiene rubber from Firestone Polymers. Budene 1207 is a high cis-butadiene rubber from Goodyear. Couplant is specified in TABLE 3. CBS is N-cyclohexyl-2-benzothiazylsulfenamide. Flexzone 7P is N-(1,3-dimethyl butyl)-N′-phenyl-p-phenylenediamine from Uniroyal.

The additives are added at a phr level (parts by weight per hundred parts by weight rubber) according to the above alternatives to a carbon black/MAA filled rubber formulation intended for tires to improve the combination of rolling resistance and wet traction. Dynamic hysteretic behavior was measured and recorded in TABLE 3:

TABLE 3
Hysteretic behavior of maleic anhydride-treated carbon black with coupler.

| | | | | | Tan delta | |
|---|---|---|---|---|---|---|
| Example | Carbon/ | | tan delta | tan delta | 0°/tan | |
| No. | Reactant | Coupler | 0° C. | 60° C. | delta 60° | Mixing Method |
| control for | N234 | none | 0.413 | 0.33 | 1.25 | CB/no coupler |
| items | | | | | | |
| 20-26 | | | | | | |
| 20 | Example 19 | | 0.347 | 0.24 | 1.45 | pre-reacted, |
| | | | | | | C/MA/Ex 18 @ |
| | | | | | | 84 phr |
| 21 | Example 8 | Structure of | 0.319 | 0.229 | 1.39 | pre-treat |
| | | Example 18 | | | | |
| | | (5 phr) | | | | |
| 22 | Example 8 | Structure of | 0.34 | 0.225 | 1.51 | pre-treat |
| | | Example 18 | | | | |
| | | (4 phr) | | | | |
| 23 | Example 8 | Structure of | 0.339 | 0.248 | 1.37 | pre-treat |
| | | Example 18 | | | | |
| | | (3 phr) | | | | |
| 24 | Example 8 | none | 0.373 | 0.322 | 1.16 | CB/MA no |
| | | | | | | coupler |
| Control | N234 | none | 0.361 | 0.289 | 1.25 | CB/no coupler |
| for item | | | | | | |
| 25 | | | | | | |
| 25 | Example 2 | Structure of | 0.299 | 0.211 | 1.42 | pre-treat |
| | | Example 18 | | | | |
| | | (4 phr) | | | | |
| Control | N234 | none | 0.372 | 0.274 | 1.36 | CB/no coupler |
| for item | | | | | | |
| 26 | | | | | | |
| 26 | Example 4 | Structure of | 0.356 | 0.23 | 1.55 | dry mix |
| | | Example 18 | | | | |
| | | (4 phr) | | | | |

Tan delta @60° C. is a measure of roll resistance; lower is better. Tan delta @ 0° C. is a measure of wet slid resistance, higher is better. Tan delta 0′/tan delta 60° C. indicates by a higher number that gains in wet skid are being achieved without equivalent losses in rolling resistance or that reduction is rolling resistance is being achieved without an equivalent loss in wet skid resistance.

The results in Table 3 show that all of the treated carbon black materials used with coupler exhibit superior dynamic properties to non-treated controls, either in a “dry mix” or by pre-reacting the CB/MA with coupler.

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

What is claimed is:

1. A filtration media comprising the reaction product of (a) a solid form of carbon and (b) a reactant of the structure
X₁X₂C═CX₃X₄, FORMULA I
or the structure
X₁C(H)═O FORMULA II
and, optionally c) a continuous media, wherein X₁, X₂, X₃ and X₄ are independently selected from H, an alkyl of 1 to 4 carbons, or an electron withdrawing group, wherein at least one of X₁, X₂, X₃ and X₄ is a known electron withdrawing group, said electron withdrawing groups being characterized by having a σₚ>0. where σₚ is the log(K′/K₀′) where K′ is the equilibrium constant for the ionization of a para substituted benzoic acid with the particular group and K₀′ is the equilibrium constant for the ionization of benzoic acid in water at 25° C., wherein said reaction product of (a) a solid form of carbon and (b) said reactant of Formula I or Formula II functions as a filtration media.

2. The filtration media of claim 1 wherein said solid form of carbon is carbon black.

3. The filtration media of claim 2 wherein said reactant is maleic anhydride or a diels alder adduct of a diene or polyene with maleic acid or anhydride.

4. The filtration media of claim 3 wherein the reaction conditions includes a temperature of at least 200° C. for at least 2 hours.

5. The filtration media of claim 1, wherein said electron withdrawing group of said reactant of FORMULA I is selected from a carboxylic acid, anhydride of carboxylic acids, acyl halide, ester, amide, nitrile, keto and aldehyde group.

6. The filtration media of claim 2 wherein said reactant is itaconic acid, glyoxylic acid, a glyoxylate ester, or a itaconic acid ester.

7. The filtration media of claim 1 wherein said solid form of carbon is graphite.

8. The filtration media of claim 7 wherein said reactant is maleic anhydride.

9. The filtration media of claim 8 wherein the reaction conditions includes a temperature of at least 200° C. for at least 2 hours.

10. The filtration media of claim 1 wherein said reaction product is further reacted with a nucleophile of the formula: R′-Nu, wherein R′-Nu contains one or more of any group known to react with said electron withdrawing groups which is at least one of X₁, X₂, X₃ and X₄, said nucleophilic groups including —NH₂, —NHR, —NR₂, —OH, —SH, —SR, —PR₂, —OP(OR)₃, —NRNHR, —NRNR₂, —NROR, or —OOR, or any anionic form thereof, and wherein R and R′ independently are hydrogen, a hydrocarbyl group, including a polyether or polyamine group, a cation-containing group, di, tri or polysulfide linkages or combinations thereof.

11-49. (canceled)

50. A filtration process comprising contacting a modified carbon substrate in media to a) remove a component, b) add a component, or c) exchange a component from the media wherein said modified carbon substrate comprises
reaction product of (a) a solid form of carbon and (b) a reactant of the structure
X₁X₂C═CX₃X₄, FORMULA I
or the structure
X₁C(H)═O FORMULA II
and, optionally c) a continuous media, wherein X₁, X₂, X₃ and X₄ are independently selected from H, an alkyl of 1 to 4 carbons, or an electron withdrawing group, wherein at least one of X₁, X₂, X₃ and X₄ is a known electron withdrawing group, said electron withdrawing groups being characterized by having a σₚ>0. where σₚ is the log(K′/K₀′) where K₀′ is the equilibrium constant for the ionization of a para substituted benzoic acid with the particular group and K₀′ is the equilibrium constant for the ionization of benzoic acid in water at 25° C.

51. A filtration process according to claim 50, wherein said solid form of carbon comprises carbon black.

52. A filtration process according to claim 50, wherein said reactant is maleic anhydride or a diels alder adduct of a diene or polyene with maleic acid or anhydride.

53. A filtration process according to claim 50, wherein the reaction conditions includes a temperature of at least 200° C. for at least 2 hours.

54. A filtration process according to claim 50, wherein said electron withdrawing group of said reactant of FORMULA I is selected from a carboxylic acid, anhydride of carboxylic acids, acyl halide, ester, amide, nitrile, keto and aldehyde group.

55. A filtration process according to claim 50, wherein said reactant is itaconic acid, glyoxylic acid, a glyoxylate ester, or a itaconic acid ester.

56. A filtration process according to claim 50, wherein said solid form of carbon is graphite or activated carbon.

57. A filtration process according to claim 50, wherein said reaction product is further reacted with a nucleophile of the formula: R′-Nu, wherein R′-Nu contains one or more of any group known to react with said electron withdrawing groups which is at least one of X₁, X₂, X₃ and X₄, said nucleophilic groups including —NH₂, —NHR, —NR₂, —OH, —SH, —SR, —PR₂, —OP(OR)₃, —NRNHR, —NRNR₂, —NROR, or —OOR, or any anionic form thereof, and wherein R and R′ independently are hydrogen, a hydrocarbyl group, including a polyether or polyamine group, a cation-containing group, di, tri or polysulfide linkages or combinations thereof.
